(19) United States
(12) Patent Application Publication
(10) Pub. No.: US 2002/0076469 A1
(43) Pub. Date: Jun. 20, 2002

(54) Composition and method

(75) Inventors: Steven Curtis Zicker, Lawrence, KS (US); Karen J. Wedekind, Meriden, KS (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 09/922,632

(22) Filed: Aug. 6, 2001

Related U.S. Application Data
(60) Provisional application No. 60/244,510, filed on Oct. 31, 2000.
(60) Provisional application No. 60/253,446, filed on Nov. 28, 2000.

(51) Int. Cl. A23L 1/30; A61K 31/385; A61K 31/355

(52) U.S. Cl. 426/72; 426/73; 514/440; 514/456; 514/474

(57) ABSTRACT

A companion pet diet meeting ordinary nutritional requirements for an aged pet and further comprising a sufficient amount of antioxidant or mixture thereof, to inhibit the deterioration of the mental capacity of an aged companion pet.

### BACKGROUND OF THE INVENTION

Companion animals such as dogs and cats seem to suffer from aging problems. Some of these are manifested in commonplace sayings. One of these is “You can't teach an old dog new tricks”. This saying arises from the observation that as dogs age, their mental capacity seems to diminish as well as physical abilities. Mental activities associated with thinking learning and memory seem to be lessened (Cummings B J, Head E, Ruehl W, Milgram N W Cotman C W 1996: The canine as an animal model of human aging and dementia; Neurobiology of aging 17:259-268). Additionally, behavioral change can be manifested in the aging animals in association with the changes in mental capacity. Many causes have been assigned to this lessening of capacity.

It has now been demonstrated that the presence of significant levels of at least one antioxidant in the diet of an aged companion pet inhibits the deterioration of the mental capacity of an aging companion pet.

### SUMMARY OF THE INVENTION

In accordance with the invention, there is a companion pet diet meeting ordinary nutritional requirements of an aged pet and further comprising a sufficient amount of an antioxidant or mixtures thereof to inhibit the deterioration of the mental capacity of an aged companion pet.

A further aspect of the invention is a method for inhibiting the deterioration of the mental capacity of an aged companion pet, which comprises feeding the pet a diet having a level of an antioxidant or mixtures thereof to accomplish this inhibition.

In further accordance with the invention is a companion aged pet diet meeting ordinary nutritional requirements of the aged pet and further comprising an antioxidant selected from the group consisting of Vitamin E, vitamin C, alpha-lipoic acid, 1-carnitine and any mixture thereof in quantities sufficient to inhibit the deterioration of the mental capacity of an aged companion pet.

A still further aspect of the invention is a method for increasing the mental capacity of an aged companion pet, which comprises feeding the aged pet an amount of an antioxidant or mixture thereof sufficient to increase the mental capacity.

In all of these methods, it is desirable to administer the antioxidant or mixture thereof in the diet of the animals.

### DETAILED DESCRIPTION OF THE INVENTION

The diet fed to the aging companion pet, for example canine and feline is the standard normal diet fed to an animal of that age. Below is a typical diet for a canine of at least 7 years of age.

TABLE 1

| Component | Target |
|---|---|
| Protein (% of dry matter) | 19.5 |
| Fat (% of dry matter) | 10 |
| Phosphorous (% of dry matter) | 0.5 |
| Sodium (% of dry matter) | 0.2 |

Adding significant quantities of an antioxidant and mixtures thereof to the companion pet diet can bring about significant and demonstrative changes in the behavior, particularly the mental capacity, as specifically shown by problem-solving capacity, in an aged pet. The term, aged, is intended to mean, in general, a canine of at least seven years and a feline of at least seven years.

The loss of mental capacity for canines and felines has been observed for a number of years. This loss of mental capacity is manifested in numerous ways. For a canine, for example, it can be manifested as disorientation, house soiling, altered sleep-wake patterns, decreased interaction with family members and pets, and inability to learn or concentrate. These conditions can be manifested in felines as well. Alzheimer's, as exhibited in man, is not found in canines and felines.

Many theories have been advanced for this loss in mental capacity. To date, the inventors are unaware of any dietary course of action, which inhibits this loss of mental capacity or can actually bring about a positive change in mental capacity as measured by an objective parameter.

The inventors have succeeded in accomplishing this. By using the diet of their invention, it has been demonstrated that aging dog's deteriorating mental capacity can be inhibited and, as measured by problem-solving capability can be enhanced. Essentially the deterioration of mental capacity can be reversed. The mental capacity of an aged pet in need of such treatment can have its mental capacity increased. Problem-solving, as demonstrated by memory and learning ability can be improved. Overall mental alertness can be enhanced. Age related cognitive decline can be slowed. With respect to Cognitive Dysfunction Syndrome, its progress can be slowed in aged dogs and clinical signs associated with this syndrome can be controlled. Prophylaxis where appropriate and pets in need of these component(s) are the target group.

The component in the diet, which accomplishes this, is an antioxidant or mixture thereof. An antioxidant is a material that quenches a free radical. Examples of such materials include foods such as Ginkgo Biloba, citrus pulp, grape pomace, tomato pomace, carrot and spinach, all preferably dried as well as various other materials such as beta-carotene, selenium, coenzyme Q10 (ubiquinone), lutein, tocotrienols, soy isoflavones, S-adenosylmethionine, glutathione, taurine, N-acetylcysteine, Vitamin E, Vitamin C, alpha-lipoic acid, 1-carnitine and the like. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The d form is preferable but racemic mixtures are acceptable. The forms and derivatives will function in a Vitamin E like activity after ingestion by the pet. Vitamin C can be administered in this diet as ascorbic acid and its various derivatives thereof such as calcium phosphate salts, cholesteryl salt, 2- monophosphate, and the like which will function in a vitamin C like activity after ingesting by the pet. They can be in any form such as liquid, semisolid, solid and heat stable form. Alpha-lipoic acid can be administered into the diet as alpha lipoic acid or as a lipoate derivative as in U.S. Pat. No. 5,621,117, racemic mixtures, salts, esters or amides thereof. L-carnitine can be administered in the diet and various derivatives of carnitine such as the salts such as the hydrochloride, fumarate and succinates, as well as acetylated carnitine, and the like can be used.

The quantities administered in the diet, all as wt % (dry matter basis) of the diet, are calculated as the active material, per se, that is measured as free material. The maximum amounts employed should not bring about toxicity. At least about 100 ppm or at least about 150 ppm of Vitamin E can be used. A preferred range of about 500 ppm to about 1,000 ppm can be employed. Although not necessary a maximum of about 2000 ppm or about 1500 ppm is generally not exceeded. With respect to Vitamin C at least about 50 ppm is used, desirably at least about 75 ppm and more desirably at least about 100 ppm. A nontoxic maximum can be employed. The quantity of alpha-lipoic acid can vary from at least about 25 ppm, desirably at least about 50 ppm, more desirably about 100 ppm. Maximum quantities can vary from about 100 ppm to 600 ppm or to an amount which remains non toxic to the pet. A preferred range is from about 100 ppm to about 200 ppm. For 1-carnitine about 50 ppm, desirably about 200 ppm, more desirably about 300 ppm for canines are a useful minimum. For felines, slightly higher minimums of 1-carnitine can be employed such as about 100 ppm, 200 ppm, and 500 ppm. A nontoxic maximum quantity can be employed, for example, less than about 5,000 ppm. For canines, lower quantities can be employed, for example, less than about 5,000 ppm. For canines a preferred range is about 200 ppm to about 400 ppm. For felines a preferred range is about 400 ppm to about 600 ppm.

Beta-carotene at about 1-15 ppm can be employed.

Selenium at about 0.1 up to about 5 ppm can be employed.

Lutein at least about 5 ppm can be employed.

Tocotrienols at least about 25 ppm can be employed.

Coenzyme Q10 at least about 25 ppm can be employed.

S-adenosylmethionine at least about 50 ppm.

Taurine at least about 1000 ppm can be employed.

Soy isoflavones at least about 25 ppm can be used.

N-acetylcysteine at least about 50 ppm can be used.

Glutathione at least about 50 ppm can be used.

Ginkgo Biloba at least 50 ppm of extract or 1% of diet can be used.

The following are raw ingredients that are high in ORAC (Oxygen radical absorbing capacity) content. When added to the diet at 1% inclusions (for a total of 5% substitution for a low ORAC ingredient such as corn) they increased the ORAC content of the overall diet and increased the ORAC content of the plasma of the animals which ate the diet containing these components. Preferably, any ingredient with an ORAC content >25 umole of Trolox equivalents per gram of dry matter could be used if added at 1% combination with four other 1% ingredients for a total of 5% addition to the diet.

Spinach pomace

Tomato pomace

Citrus Pulp

Grape pomace

Carrot granules

Broccoli

Green tea

Ginkgo Biloba

Corn gluten meal

### EXAMPLE 1

All dogs were beagles and 7 years old or greater. The nutritional components of the control and test diet were approximately the same as the typical diet disclosed earlier in Table 1. However, the control diet contained 59 ppm Vitamin E and <32 ppm Vitamin C. The test diet had 900 ppm Vitamin E and 121 ppm Vitamin C, 260 ppm 1-carnitine and 135 ppm alpha lipoic acid.

Twelve--aged beagle dogs were given a battery of baseline problem solving tasks prior to placement into either a control or enriched test diet group. The aged animals were equally matched with respect to learning (discrimination reversal) and memory (delayed non-match to position [DNMP] and delayed non-match to sample [DNMS]). A T-test was used to compare the two groups of dogs on baseline learning of the discrimination reversal learning, DNMP, and DNMS tasks. The results were non-significant . Thus, dogs were equally matched on the basis of cognition prior to diet intervention. Approximately 1 month after starting the diet, the first problem-solving task given to dogs was a landmark discrimination learning task, which is a test of spatial attention (Milgram et al., 1999 Milgram, N. W., Adams, B., Callahan, H., Head, E., Mackay, B., Thirlwell, C., & Cotman (1999), C. W. Landmark Discrimination Learning in the Dog. Learning & Memory, 6:54-61).

Landmark discrimination learning requires subjects to select a particular object based on proximity to an object. The initial learning, however, is based on the dogs' ability to learn an object discrimination task. We have previously found that the effects of age on discrimination learning depends on task difficulty, and we have evidence to indicate that landmark discrimination learning is markedly impaired in aged dogs.

When aged animals on the enriched test diet and control diet were compared on the landmark discrimination learning tasks, there was a highly significant difference between the groups. (p<02). Animals on the enriched diet acquired the task with fewer errors than did the animals on the control diet. Whereas all 6 of the animals on the enhanced diet were able to meet the learning criterion within 40 sessions, only 3 of the 6 animals on the control diet were able to meet the learning criterion. In addition, the 3 dogs that were able to solve the problem committed more errors than dogs receiving the enriched diet.

Dogs in the control and enriched test diet group, after completing landmark discrimination learning, have been tested on an oddity task. This task involves presenting dogs with 3 objects covering all 3 food wells. Two of these objects are identical and one is different. To obtain a food reward, dogs must select the odd object. Dogs on the enriched test diet learned this task with significantly fewer errors than dogs fed the control diet (p<0.003 for all 4 oddity test scores combined).

### EXAMPLE 2

Beagles (n=28) were pre-trained on a size discrimination task and ranked according to the errors to criteria in learning this task. The dogs were then stratified by rank into groups of three and randomly assigned to one of three diets based on prior cognition scores. All dogs enrolled in this study were greater than 7 years of age. Dogs were placed on one of three dry foods varying in vitamin E content and initiated on a landmark discrimination protocol. The Vitamin E content and other components are listed in Table 2 below.

TABLE 2

| Diet No. | Vitamin E | Vitamin C | L-Carnitine | Lipoic Acid |
|---|---|---|---|---|
| 1 | 799 ppm | 114 ppm | 294 ppm | 135 ppm |
| 2 | 172 ppm | <32 ppm | 42 ppm | None added |
| 3 | 57 ppm | <32 ppm | 13 ppm | None added |

The landmark discrimination protocol consisted of three phases of testing (landmark 0, 1, 2) which required dogs to reach a passing criteria (8/10 correct for two days in a row followed by 7/10 average for next three days) before moving to the next phase of the test. Each dog was allowed 40 days with 10 trials per day to learn each phase. Repeated MANOVA revealed a significant overall effect of diet on errors to criteria scores (P<0.05). Regression analysis of the summation of errors for landmark 1+2 versus the Vitamin E content of the diet revealed a significant (P<0.05) regression slope with dogs on the highest E diet making the least errors (mean=65) and those on the lowest E diet making the most errors (mean=170).

What is claimed is:

1. A companion pet diet meeting ordinary nutritional requirements for an aged pet and further comprising a sufficient amount of an antioxidant or mixture thereof, to inhibit the deterioration of the mental capacity of an aged companion pet.

2. The diet in accordance with claim 1 wherein the pet is a canine.

3. The diet in accordance with claim 2 wherein the canine is at least seven years.

4. The diet in accordance with claim 1 wherein the pet is a feline.

5. The diet in accordance with claim 4 wherein the feline is at least seven years.

6. The diet in accordance with claim 1 wherein Vitamin E is present in at least about 100 ppm of the diet.

7. The diet in accordance with claim 6 wherein an antioxidant selected from the group consisting of Vitamin C, 1-carnitine, alpha-lipoic acid or mixtures thereof is present in the diet.

8. The diet of claim 1 wherein an antioxidant selected from the group consisting of Vitamin C, 1-carnitine, alpha-lipoic acid or mixture thereof is present in the diet.

9. The diet of claim 8 wherein at least about 50 ppm of Vitamin C are in the diet.

10. The diet of claim 8 wherein at least about 25 ppm of alpha-lipoic acid are in the diet.

11. The diet of claim 8 wherein at least about 50 ppm of 1-carnitine are present in the diet.

12. A method for inhibiting the deterioration of the mental capacity of an aged companion pet which comprises feeding the pet a level of antioxidant or mixture thereof to accomplish this inhibition.

13. The method in accordance with claim 12 wherein the pet is a canine.

14. The method in accordance with claim 13 wherein the canine is at least seven years.

15. The method in accordance with claim 12 wherein the pet is a feline.

16. The method in accordance with claim 15 wherein the feline is at least seven years.

17. The method in accordance with claim 12 wherein Vitamin E is fed the pet in at least about 100 ppm as measured by the diet.

18. The method in accordance with claim 17 wherein an antioxidant selected from the group consisting of Vitamin C, alpha-lipoic acid 1-carnitine or mixtures thereof is fed the pet.

19. The method of claim 12 wherein amounts of Vitamin C, 1-carnitine, alpha-lipoic acid or mixture thereof are fed the pet.

20. The method of claim 19 wherein at least about 50 ppm of Vitamin C is fed the pet.

21. The method of claim 19 wherein at least about 25 ppm of alpha-lipoic acid is fed the pet.

22. The method of claim 19 wherein at least about 50 ppm of 1-carnitine is fed the pet.

23. A companion pet diet meeting ordinary nutritional requirements of an aged pet and further comprising at least about 100 ppm of Vitamin E, at least about 50 ppm of Vitamin C, at least about 25 ppm of alpha-lipoic and at least about 50 ppm of 1-carnitine in the diet.

24. The diet in accordance with claim 23 wherein the aged pet is a canine of at least 7 years.

25. The diet in accordance with claim 23 wherein the aged pet is a feline of at least seven years.

26. A method for increasing the mental capacity of an aged companion pet which comprises feeding the pet a diet having a sufficient amount of antioxidant or mixture thereof to accomplish the increase.

27. The method in accordance with claim 26 wherein the pet is a canine.

28. The method in accordance with claim 27 wherein the canine is at least seven years.

29. The method in accordance with claim 26 wherein the pet is a feline.

30. The method in accordance with claim 29 wherein the feline is at least seven years.

31. The method in accordance with claim 26 wherein the Vitamin E is fed the pet in at least about 100 ppm as measured by the diet.

32. The method in accordance with claim 31 wherein an antioxidant selected from the group consisting of Vitamin C, alpha-lipoic acid, 1-carnitine or mixture thereof is fed the pet.

33. The method of claim 26 wherein amounts of Vitamin C, 1-carnitine, alpha-lipoic acid or mixture thereof are fed the pet.

34. The method of claim 33 wherein at least about 50 ppm of Vitamin C is fed the pet.

35. The method of claim 33 wherein at least about 25 ppm of alpha-lipoic acid is fed the pet.

36. The method of claim 33 wherein at least about 50 ppm of 1-carnitine is fed the pet.

37. A companion pet diet meeting the nutritional requirements and having enough antioxidant or mixture thereof to increase the mental capacity of an aged pet.
